(19) United States
(12) Patent Application Publication
(10) Pub. No.: US 2021/0236537 A1
(43) Pub. Date: Aug. 5, 2021

(54) HIGH POROSITY CELLULOSIC STRUCTURES AND METHODS OF TREATMENT THEREWITH

(71) Applicant: BioLumen Inc., Sausalito, CA (US)

(72) Inventors: Paolo Costa, Sausalito, CA (US); Gabriel Paulino, Sausalito, CA (US)

(21) Appl. No.: 17/166,991

(22) Filed: Feb. 3, 2021

Related U.S. Application Data
(60) Provisional application No. 62/969,453, filed on Feb. 3, 2020.

(51) Int. Cl. A61K 31/717; A61K 9/28; A61P 1/00

(52) U.S. Cl. CPC A61K 31/717; A61P 1/00; A61K 9/28

(57) ABSTRACT

The present disclosure is directed to compositions of materials and methods of making those compositions. These compositions include cellulose materials that may be consumed by an animal without chewing. Compositions of the present disclosure may also include coatings that resist dissolution in the mouth of an animal, yet readily dissolve in the digestive tract of an animal or human/person. The present disclosure is also directed to administering selected compositions to treat specific ailments or conditions that may affect animals or humans. Such treatments include treating conditions related to improving gut health, reducing neuro-inflammation, and treating metabolic diseases. Additionally, such treatments include the stimulation, enhancement and/or compatibility with microbiota and the effects of microbiota on diseases and conditions, such as gut health of an animal, including a human.

### CROSS-REFERENCE TO RELATED APPLICATIONS

The present application claims priority benefit of provisional patent application 62/969,453 filed Feb. 3, 2020, the disclosure of which is incorporated herein by reference.

### BACKGROUND OF THE INVENTION

### Field of the Invention

The present disclosure generally relates to the compositions and methods of using such compositions for treatment of conditions suffered by an animal or human. More specifically the present disclosure is directed to treating diseases conditions including those may be associated with the digestive tract of an animal or person.

### Description of the Related Art

Various diseases and conditions are related to excess amounts of absorption of sugars and salts in the digestive system, or gut of animals and people. Such conditions include diabetes, obesity, metabolic syndromes, non-alcoholic fatty liver disease (NAFLD), chronic inflammation, and neuro-degenerative conditions related to neuro-inflammation

Although various therapies exist for a variety of diseases and conditions including those mentioned above, there remains a need to find better, more efficacious, safer, more cost-effective and more accessible treatments. There is also a need for new compositions of matter that can be used to improve overall health of people and animals.

### SUMMARY OF THE PRESENTLY CLAIMED INVENTION

The presently claimed invention is directed to methods for making and administering compositions of materials to treat various diseases or conditions. In a first embodiment, a method for treating a gut disease or condition includes providing a consumable high porosity cellulosic structure for consumption by an animal. Once consumed by the animal, the high porosity cellulosic structure stimulates the growth of one or more microorganisms in a gut of the animal.

In a second embodiment, a method for treating a disease or condition includes providing a consumable high porosity cellulosic structure to an animal for consumption. Once consumed by the animal, the high porosity cellulosic structure reduces the amount of an available component in the gut of the animal, the available component including at least one of a fat, a sugar, salt, or a high caloric food substance.

A third presently claimed embodiment is a composition of matter comprising a high porosity cellulosic structure prepared to a size capable of being capable of being directly swallowed by an animal. After prepared high porosity cellulosic structure is consumed by an animal, pores in the high porosity cellulosic structure receive and entrap one or more compounds in a gut of the animal.

### BRIEF DESCRIPTION OF THE DRAWINGS

FIG. 1 illustrates glucose absorption data as compared to a control group for each of a first treatment using tablets of a first type and a second treatment using tablets of a second type.

FIG. 2 illustrates fructose absorption data as compared to a control group for each of the first and the second treatment.

FIG. 3 illustrates sucrose absorption data as compared to a control group for each of the first and the second treatment.

FIG. 4 illustrates experimental data associated with digestion of starch in the upper part of a gastro intestinal (GI) tract of an animal during a GI tract experiment.

FIG. 5 illustrates starch to glucose conversion at the end of the duodenum during the GI tract experiment.

FIG. 6 illustrates data related to the starch to glucose conversion at the end of the small intestine when the different treatments (1 and 2) were compared against a standard control of the GI tract experiment.

FIG. 7 illustrates pH decrease in the colon of an animal as compared to a control group in a cellulose tablet-gut-microbiome-experiment.

FIG. 8 illustrates gas pressure in colon of an animal as compared to the control group in the cellulose tablet-gut-microbiome-experiment.

FIG. 9 illustrates acetate production in the animal colon as compared to the control group in the cellulose tablet-gut-microbiome-experiment.

FIG. 10 illustrates propionate production in the animal colon as compared to the control group in the cellulose tablet-gut-microbiome-experiment.

FIG. 11 illustrates butyrate production in the animal colon as compared to the control group in the cellulose tablet-gut-microbiome-experiment.

FIG. 12 illustrates lactate production and consumption in the animal colon as compared to the control group in the cellulose tablet-gut-microbiome-experiment.

FIG. 13 illustrates branched short chain fatty acids production in the animal colon as compared to the control group in the cellulose tablet-gut-microbiome-experiment.

FIG. 14 illustrates steps that may be performed when a high porosity cellulose material is prepared for consumption by an animal.

### DETAILED DESCRIPTION

The present disclosure is directed to compositions of materials and methods of making those compositions. These compositions include cellulose materials that may be consumed by an animal without chewing. Compositions of the present disclosure may also include coatings that resist dissolution in the mouth of an animal or person/human, yet readily dissolve in the digestive tract of an animal or human. The present disclosure is also directed to administering selected compositions to treat specific ailments or conditions that may affect animals or humans. Such treatments include treating conditions related to improving gut health, reducing neuro-inflammation, and treating metabolic diseases. Additionally, such treatments include the stimulation, enhancement and/or compatibility with microbiota and the effects of microbiota on diseases and conditions, such as gut health of an animal, including a human.

The compositions discussed herein include a consumable high porosity cellulosic structure that when present in the gut environment of an animal that will entrap sugars and starches present in the gut. Such starches when entrapped in the structure from a resistant starch or a partially resistant starch. The resistant starch is then accessible to microbiota present in the gastrointestinal (GI) tract, particularly in the lower parts of the GI tract, and are able to reach the resistant starch within the high porosity cellulosic structure. The compositions permit small sugars (e.g., sucrose, fructose and glucose) to diffuse out of the structure. The compositions provided herein stimulate the growth of one or more microorganisms in the animal GI tract and thereby contribute to gut health of the animal. These compositions may reduce, control, or otherwise limit the digestion or absorption of entrapped sugars or starches after they have been ingested or consumed by an animal.

As used herein “animal” includes mammals, such as humans, companion animals (e.g., dogs and cats), and livestock animals (e.g., cows, pigs, goats, sheep).

As used herein “consumable” means that the composition (e.g., the high porosity cellulosic structure) can be safely consumed (e.g., eaten or otherwise ingested) by an animal.

In some embodiments, the starches trapped within the high porosity cellulosic structure or a portion thereof are converted to short chain fatty acids by one or more microorganisms resident in the GI tract. Such short chain fatty acids include, for example, acetate, propionate, butyrate, lactate or any combination thereof. In some embodiments, the starches trapped within the high porosity cellulosic structure or the high porosity cellulosic structure itself are not converted to or does not substantially increase the amount of branched chain fatty acids produced by the microorganisms. In some embodiments, the starches trapped within the high porosity cellulosic structure itself do not substantially increase the amount of protein fermentation in the animal gut.

In some embodiments, the compositions provided herein are administered in conjunction with a probiotic (live beneficial microorganisms), and administered prior to, subsequent to or co-administered with a probiotic.

The compositions herein can be used for treatment. Provided herein are methods of treating a disease or condition comprising providing a consumable high porosity cellulosic structure to an animal, the cellulosic structure is capable of sequestering a sugar present in an animal gut. These compositions may sequester sugars, thereby lowering absorption of the sugar in the bloodstream of the animal. Sugars can include, but are not limited to sucrose, fructose, glucose or any combinations thereof. In some embodiments of the methods, the amount of sugar absorbed by the high porosity cellulosic structure in the stomach is between about 10% and 60% of the sugar present in the stomach of the animal. In some embodiments of the methods, the amount of sugar absorbed by the high porosity cellulosic structure is between about 7% and 30% of the sugar present in the small intestine. In some embodiments of the methods, the amount of sugar absorbed by the small intestine of the animal is reduced between about 3% and 20%.

The high porosity cellulosic structures and compositions thereof include cellulose and at least one or more of an additional component selected from carrageenan, glucomannan, carboxymethylcellulose, persimmon tannins, pectin, gellan gum, beta-glucan soluble fiber, psyllium husk, guar gum, locust bean gum, hydroxypropylmethylcellulose, mixed plant cell wall fibers, arabinoxylan, alginate, inulin and inulin-type fructans, high amylose RS2, galactooligosaccharide, polydextrose, resistant maltodextrin/dextrin, cross-inked phosphorylated RS4, and any combinations thereof. The cellulose may be coated with one or more the additional component(s). A cellulosic high porosity structure consistent with the present disclosure may include cellulose, carrageenan and glucomannan. In some instances, the cellulose is converted to cellulosic sponge and then coated with carrageenan and glucomannan. The high porosity cellulosic structure may comprises cellulose and one or more compounds selected from the group consisting of carrageenan, glucomannan and carboxymethylcellulose.

In some instances, a high porosity cellulosic structure and compositions thereof is formulated into a tablet or pill or other solid form. Such a tablet or pill may be coated such as to resist water (e.g., such as degradation in the mouth of the animal) but readily dissolves in the stomach of animal (e.g. pH lower than 5). Forms of these pills or tablets may be sufficiently small (e.g., from about 10 nanometers to about 1 millimeter) so that it can be added to food prior to its consumption by an animal. The compositions discussed herein may be incorporated into a pre-packaged form of a food, such as a human food, such as chocolate, chocolate spreads, snack bars, cheese, pasta, sauces, jams, dressings, baked foods, fried foods, roasted foods, canned foods, and spreads, or alternatively the compositions are added, sprinkled, dispersed like a powder on food just prior to consumption.

The compositions provided herein are useful for the treatment of diseases and conditions and the improvement of overall and/or specific aspects of animal health, including human health. In some embodiments, the compositions are administered for maintenance of a healthy balanced gut. In some instances, the compositions are administered to treat diabetes, such as Type I or Type II diabetes, or administered to improve the health of an individual diagnosed with and/or suffering from diabetes. The compositions may be administered to aid in the management of glucose homeostasis.

The administration of a high porosity cellulosic structure and compositions thereof may reduce the amount of an available component in the gut of the animal, such as one or more of fat, sugar, salt, and a high caloric food substance. The high porosity cellulosic structure absorbs one or more of such components from the GI tract (gut) of the animal, such as from the stomach of the animal, small intestine of the animal or a combination thereof.

The compositions may be administered for treating, ameliorating or otherwise improving the health of an animal, including a human, diagnosed with or having symptoms of metabolic syndrome, type 2 diabetes, Non-Alcoholic Fatty Liver Disease (NAFLD), chronic inflammation, and a neuro-degenerative condition related to neuro-inflammation. The neuro-inflammation condition can be one that effects the enteric nervous system and its translocation to the brain via the vagus nerve.

The following examples are included for illustrative purposes only and are not intended to limit the scope of the invention.

### EXAMPLES OF TABLET PREPARATION

Preparation of Tablet 1: A cellulosic high porosity material was coated with carrageenan and glucomannan in variable quantities and ratios. Cellulose ratio to hydrogels ratio, from 1% to 99%. Carrageenan ratio to cellulose ratio: from 1% to 99%. Glucomannan ratio to cellulose ratio: from 1% to 99%. Same ratio: 1% to 99% for any combination of two of the above components to the third one.

Alternative preparation of Tablet 1 may include the steps of:
Coating tablet 1 with persimmon tannins and/or pectin, gellan gum, beta-glucan soluble fiber, psyllium husk, guar gum, locust bean gum, hydroxypropylmethylcellulose, mixed plant cell wall fibers, arabinoxylan, alginate, inulin and inulin-type fructans, high amylose RS2, galactooligosaccharide, polydextrose, resistant maltodextrin/dextrin, cross-inked phosphorylated RS4 or a combination of any of these fibers: in proportion from 0.5% to 99.5%.
Coating cellulose with persimmon tannins and any of the carrageenan or glucomannan, in proportion from 0.5 to 99.5%.
A tablet completed made of hydrogels in a dry state and highly porous, that get ingested in a coated tablet and release in the stomach, then they absorb stomach fluids and gel them and that can resist partially, from 1% to 99%, the GI tract chemical and mechanical action and that hinder the intestinal absorption of the nutrients absorbed by the hydrogels.

Preparation of Tablet 2 may include the steps of:
1. Activate high porosity cellulosic sponge by pre-treatment in a sodium hydroxide (NaOH) solution.
2. React with chloroacetic acid.
3. Stop reaction at from 1 to 100% conversion of glucose residues in cellulose to CMC (much greater carboxymethylation makes the polymer backbone soluble in water, compromising the sponge integrity).
4. Remove high porosity cellulosic sponges from liquid reaction mixture and rinse extensively in water.
5. Reaction in hot water (per 1 g sponge dry weight).
6. Pre-treat sponges in 3M NaOH from 35 C to 90 C for at least 1 minute, maximum 7 hours.
7. Add 1 g to 5 g or any fraction between these 2 quantities of chloroacetic acid and maintain temperature stir from 1 minute to 60 minutes in a container.
8. Rinse

Delivery format of tablet 1 or 2 may use any of the above tablets, coated in order to resist water but that readily dissolve in stomach where pH lower than 5. These tablets may be of a size small enough not to be readily noticed by a person or animal consuming them. For, example, these tablets may be of a size that range from the nanometer range to the millimeter range (e.g. from 10 nanometers to 1 mm). The sizes of these tablets may be measured based on a cross-section, a diameter, a length, or a width of the tablets. These tablets may be added to a food when it is manufactured or may be added to or applied to foods before they are consumed. For example, a small spoon may be used to extract volumes of the tablets from a vial before they are added to or applied to the surface of a food. These tablets may be added to food, including pre-packaged foods, prior to its consumption by any available method. As such these tablets may be added to or mixed with chocolate, chocolate spreads, cheese, pasta, sauces, jams, dressings, baked foods, fried foods, roasted foods, canned foods, spreads, etc., or alternatively added, sprinkled, dispersed like a powder on food just prior to consumption.

### Examples of Absorption of Free Sugars in an Upper Part of the Gastro Intestinal (GI) Tract of an Animal

Tablets 1 or 2 may be administered after they have been fabricated. Any of the above tablets may have been coated in order to resist water, where that coating may readily dissolve in the stomach of an animal based on the low pH or acidic environments commonly associated with the stomach of an animal. This coating may resist dissolving in the mouth of an animal based on a pH of the animal's mouth being higher than a pH of the animal's stomach. This is because the pH of an animal's mouth is typically greater than 5 (e.g. 5.3) and because the pH of the animal's stomach is typically less than 5 (e.g. 1.5 to 3.5).

After fabrication, tablets 1 & 2 were then tested with an artificial gut model called the SHIME: Simulator of Human Intestinal Microbial Ecosystem as described in the following publication (https://link.springer.com/chapter/10.1007/978-3-319-16104-4_27). This SHIME model is composed of multiple compartments modeling the individual phases of the human digestive system including the oral, gastric (stomach), small intestine (site of absorption of nutrients) and colon (site of intensive microbial fermentation).

### Material and Methods Glucose, Fructose, & Sucrose Absorption Experiment

An amount of free sugars that was required to provide a relevant amount of kilocalories (Kcal) was calculated based on:
A caloric content of sugars=4 Kcal/gram,
A % free sugars content of normal diet=10% (fact sheet Word Health Organization),
A volume of gastric incubation=100 mL, and
A composition of sugar mixture to be tested=1:1:1 (glucose:fructose:sucrose)

Other assumptions used during the experimentation included a combination of 42 grams/liter of each sugar simulates sugar intake of a diet equivalent to 500 Kcal and a corresponding dose of cellulose tablets to be tested=1.8 gram/incubation/.

Since an oral phase implies administration of total sugar content in 20% of gastric volume, a concentration of 210 grams/Liter of each sugar was prepared and measured via HPAEC-PAD (High-performance anion exchange chromatography with pulsed amperometric detection).

It was shown that these measurements corresponded well with theoretical values. Furthermore, a first test (test 1) can be performed under optimal concentrations. An end point of the experiment was the potential sequestration of glucose, fructose, and sucrose in the cellulose tablets and subsequent inhibition of small intestinal absorption. The experiment consisted of a simulated upper gastro-intestinal incubation for:
Control (n=3)
Cellulose tablet 1 (n=3)
Cellulose tablet 2 (n=3)

After simulating a short exposure in the oral phase (where cellulose tablets were allowed to swell and take up the concentrated sugar solutions), a stomach (2 hour incubation) and small intestinal condition (3 hour 30 minute incubation) were simulated. The different phases correspond to a time food spends in the mouth, in the stomach, and in the small intestine. Absorption in the small intestine was simulated via a dialysis approach during the final 3 hours of incubation.

### Glucose, Fructose, & Sucrose Experimental Results

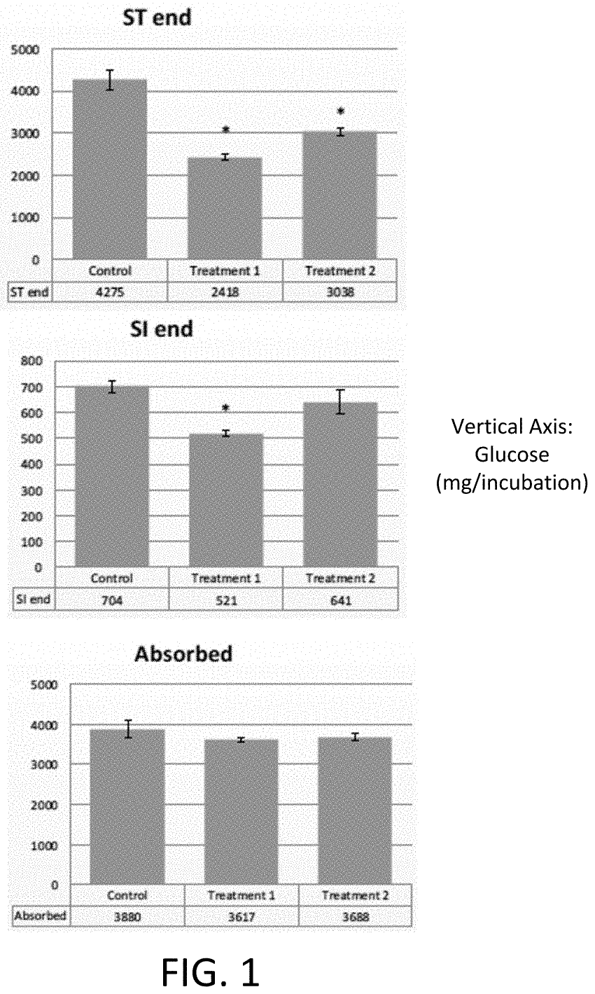

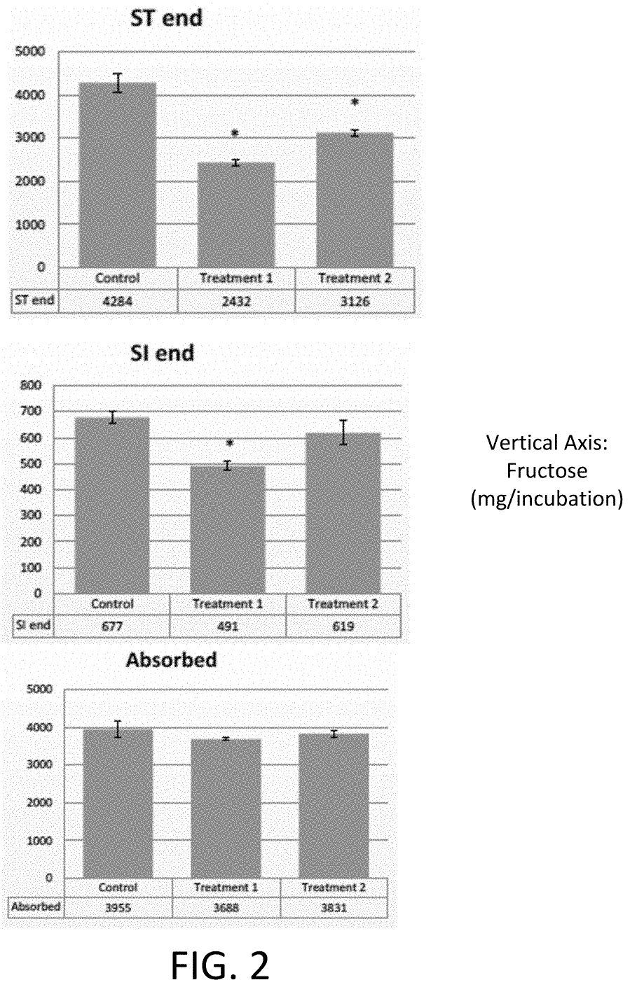

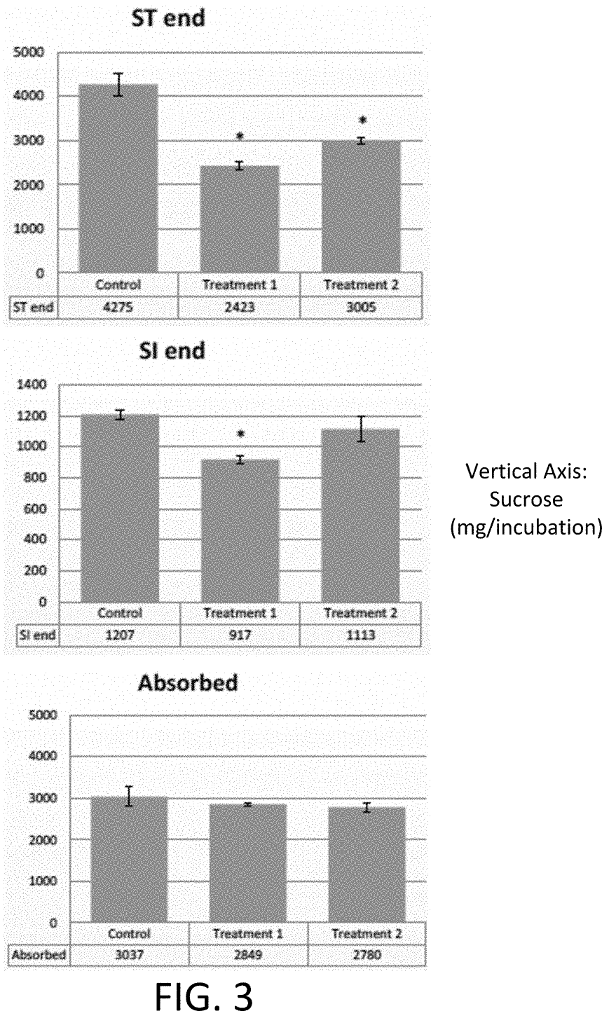

FIG. 1 illustrates glucose absorption data as compared to a control group for each of a first treatment using tablets of a first type and a second treatment using tablets of a second type. FIG. 2 illustrates fructose absorption data as compared to a control group for each of the first and the second treatment. FIG. 3 illustrates sucrose absorption data as compared to a control group for each of the first and the second treatment.

At the start of the incubation 4.2 grams (g) of glucose, 4.2 g of fructose and 4.2 g of sucrose was added the gastric volume. Each of FIGS. 1-3 include three sets of graphs, a first set of graphs “ST end,” a second set of graphs “SI end,” and a third set of graphs “Absorbed.” The “ST end” of these graphs corresponds to absorption of glucose, fructose, and sucrose absorbed by the end of the stomach of an animal. The “SI end” of these graphs corresponds to content of glucose, fructose, and sucrose at the end of the small intestine of an animal. The “Absorbed” graph illustrates amounts of glucose, fructose, and sucrose absorbed in the small intestine. This data indicates that the consumption of each of tablets 1 and tablets 2 with food resulted in a significant decrease in absorption of glucose, fructose, and sucrose in the GI tract of an animal. Numeric data from each of the respective graphs of FIGS. 1-3 may be evaluated by calculating percentage differences of data points of treatment 1 (type 1 tablet consumption) and treatment 2 (type 2 tablet consumption) as compared to a standard control. For example, a percentage (%) difference/change of glucose absorbed in the stomach under treatment 1 (“ST end” treatment 1) data as compared to control data may be calculated by the formula ([(control value)−(treatment 1 value)])/(control value))×100=% change: ([4275−2418]/4275)*100=43.4%. As such, treatment 1 reduced amounts of glucose absorbed in the stomach by over forty-three percent. A summary of the data illustrated in FIGS. 1-3 is illustrated below.
1. Cellulose tablets 1 and 2 absorbed part of the volume in the stomach (of about 40% and 30%, respectively) resulting in a decrease of the glucose/fructose/sucrose levels in the gastric content at the end of the stomach incubation with 43.4%/43.2%/43.4% and 28.9%/27.0%/29.7%, respectively (as shown in FIGS. 1, 2 & 3 under ST end).
2. Cellulose tablets 1 and 2 decreased small intestinal glucose/fructose/sucrose absorption with 6.8%/6.7%/6.2% and 4.9%/3.1%/8.5%, respectively (as shown in FIGS. 1, 2 & 3 under Absorbed).
3. Cellulose tablets 1 and 2 absorbed part of the volume in the small intestine (˜15%) resulting in a decrease of the glucose/fructose/sucrose levels in the small intestinal content at the end of the small intestinal incubation with 26.0%/27.5%/24.0% and 8.9%/8.7%/7.8%, respectively (as shown in FIGS. 1, 2 & 3 under SI end).

### Materials and Methods Starch Digestion in the Upper Part of the GI Tract Experiment

An end point of this experiment was the conversion of starch to glucose and the subsequent absorption of glucose in the small intestine. The experiment included of a simulated upper gastro-intestinal incubation. After simulating the oral phase (with salivary amylase addition), a fed state in the stomach (2 hour incubation) and small intestine (3 hour 30 minute incubation) was simulated. Absorption in the small intestine was simulated via a dialysis approach. At the start of the experiment, 850 mg of starch is added.
Control (n=3)
Cellulose tablet 1 (n=3)
Cellulose tablet 2 (n=3)

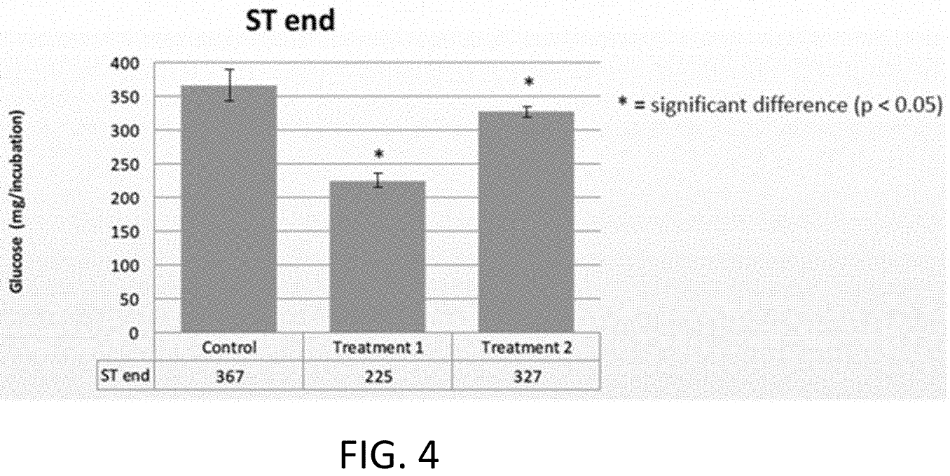

FIG. 4 illustrates experimental data associated with digestion of starch in the upper part of a gastro intestinal (GI) tract of an animal during a GI tract experiment. FIG. 4 illustrates that cellulose tablets of type 1 and 2 absorbed great part of the volume materials in the stomach resulting in a decrease of the glucose levels in the gastric content with 38.6% (p=0.0006) and 10.9% (p=0.047), respectively (as seen in graph on FIG. 4).

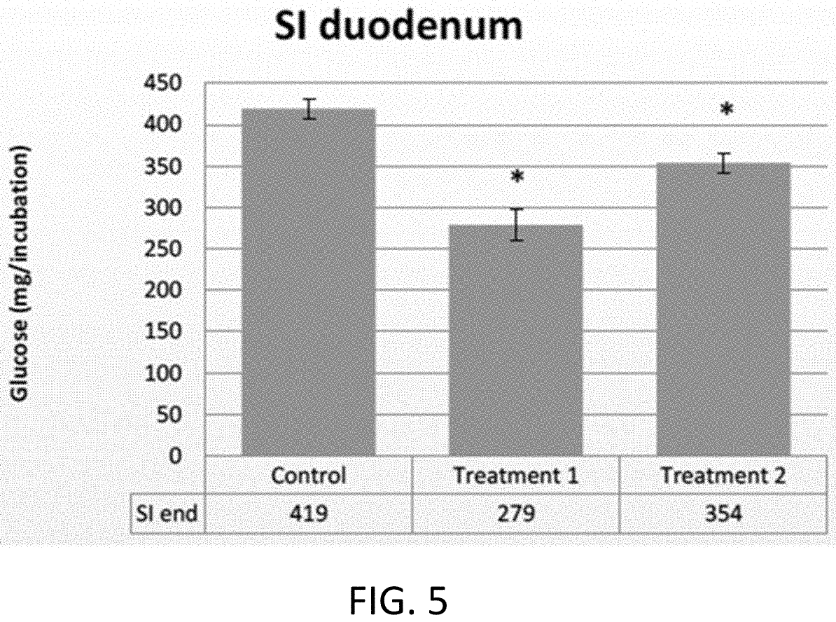

FIG. 5 illustrates starch to glucose conversion at the end of the duodenum during the GI tract experiment. This data shows that cellulose tablets 1 and 2 significantly lowered glucose levels in the duodenum with 33.4% (p=0.0004) and 15.6% (p=0.0026), respectively (as seen in graph on FIG. 5).

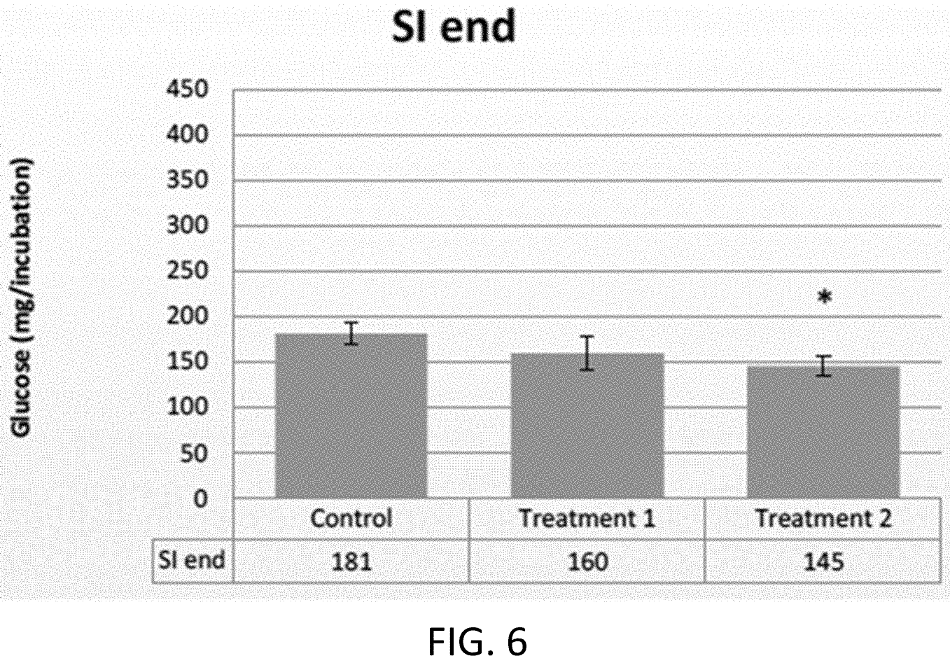

FIG. 6 illustrates data related to the starch to glucose conversion at the end of the small intestine when the different treatments (1 and 2) were compared against a standard control of the GI tract experiment. This data shows that cellulose tablet 2 significantly decreased the glucose levels in the small intestinal content (19.8% decrease; p=0.0486) at the end of the small intestinal phase (as seen in FIG. 6).

The starch to glucose experiment indicates that lower glucose levels in both gastric and duodenal content could result from: the physical up take of glucose in tablets and/or a decreased conversion of starch to glucose.

### Impact of Cellulose Tablets on Gut Microbiome in the Lower Part of the GI Tract

The aim of this cellulose tablet-gut-microbiome-experiment was to understand how the transfer of starch retained in the cellulose tablets could stimulate overall microbial fermentation by the human gut microbiome (e.g. gas production, pH decrease) by potentially increasing the production of health-related metabolites (e.g. lactate and SCFA), while decreasing the production of metabolites that are related to adverse health effects (branched SCFA). The experiment consisted of a simulated colon incubation for:
Control (n=3)
Cellulose tablet 1 (n=3)
Cellulose tablet 2 (n=3)

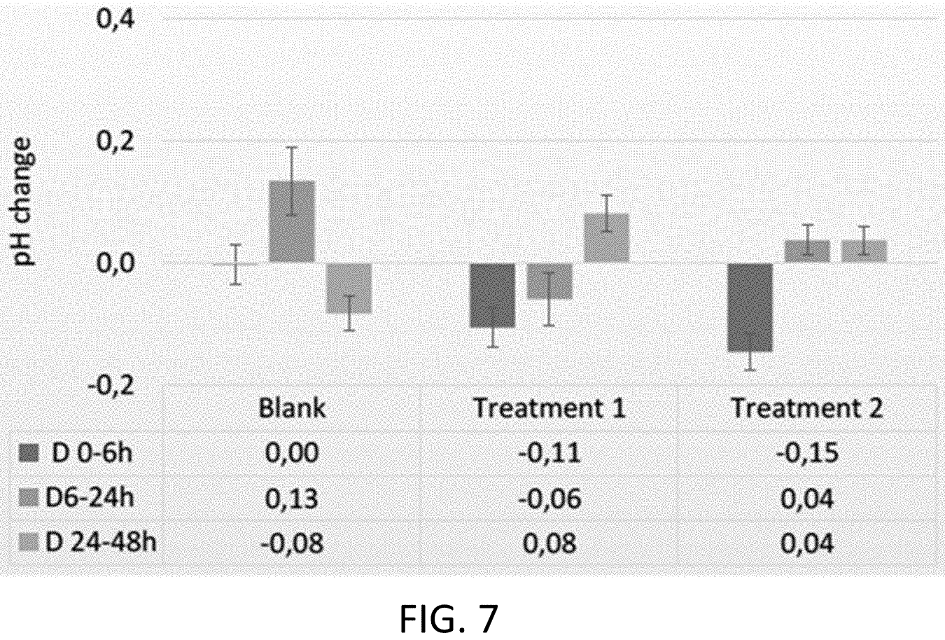

FIG. 7 illustrates pH decrease in the colon of an animal as compared to a control group in a cellulose tablet-gut-microbiome-experiment. This data shows a decrease in pH, where cellulose tablets resulted in a stronger initial pH decrease (0-6 hours) revealing a stronger initial microbial activity, potentially due to fermentation of starch by the gut microbiota (as shown in FIG. 7).

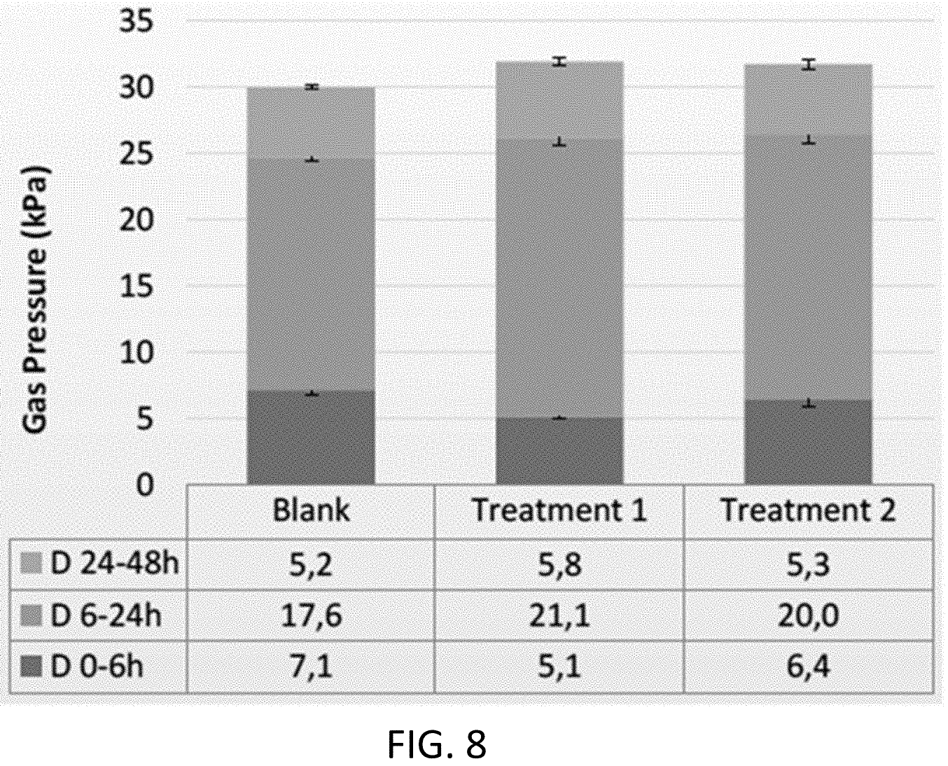

FIG. 8 illustrates gas pressure in colon of an animal as compared to the control group in the cellulose tablet-gut-microbiome-experiment. Here cellulose tablets resulted in a mild increase in overall gas production, mostly in the 6-24-hour time interval, again indicating a stimulation of the overall microbial activity, potentially due to starch fermentation (as shown in FIG. 8).

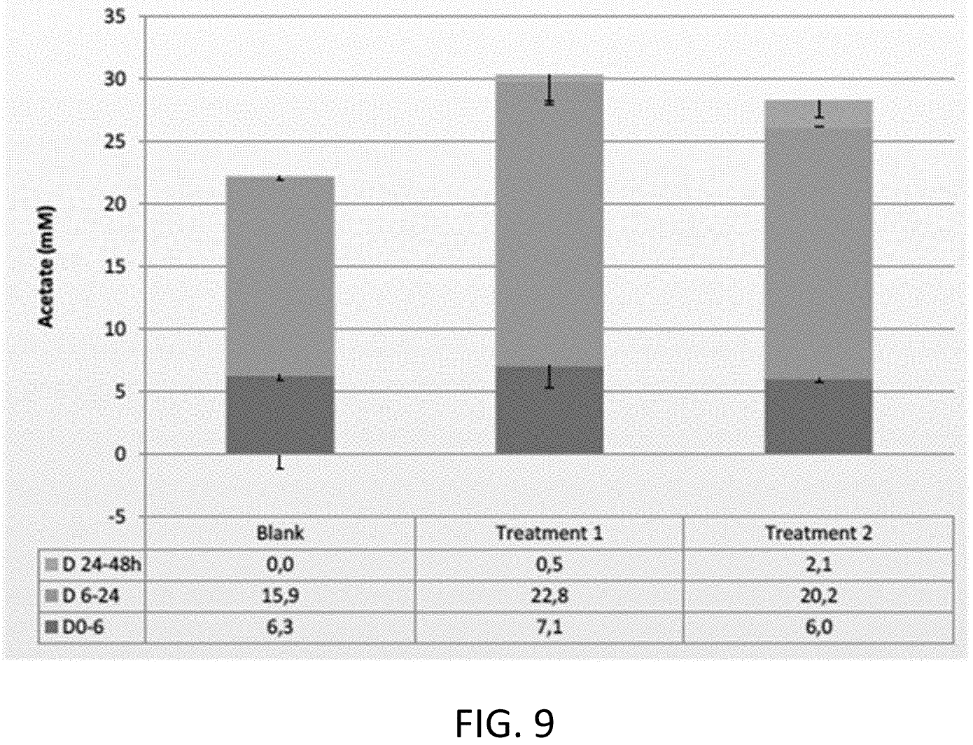

FIG. 9 illustrates acetate production in colon of the animal as compared to the control group in the cellulose tablet-gut-microbiome-experiment. This data shows cellulose tablets (especially tablet 1) strongly increased acetate production, mostly in the 6-24-hour time interval, indicating a stimulation of the overall microbial activity, potentially due to starch fermentation (as shown in FIG. 9).

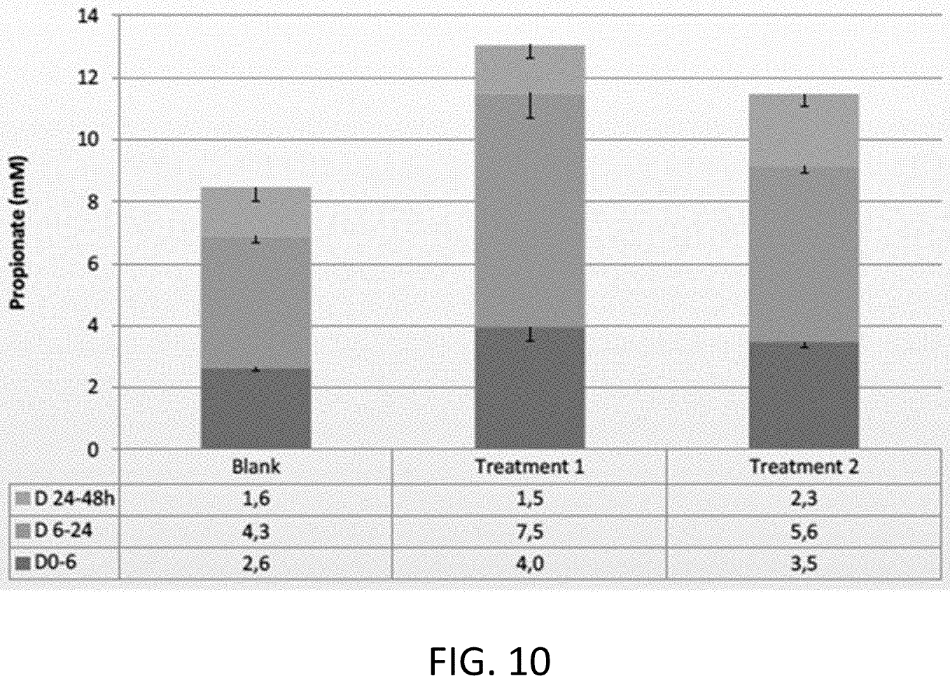

FIG. 10 illustrates propionate production in the colon of the animal as compared to the control group in the cellulose tablet-gut-microbiome-experiment. This data shows that cellulose tablets (especially tablet 1) strongly increased propionate production, mostly in the 0-24-hour time interval, indicating a stimulation of the overall microbial activity, potentially due to starch fermentation (as shown in FIG. 10). Since propionate production is associated with lowering cholesterol and oxidation of lipids, the data indicates that cellulose tables as described herein, could help control or reduce cholesterol levels in the blood of a person.

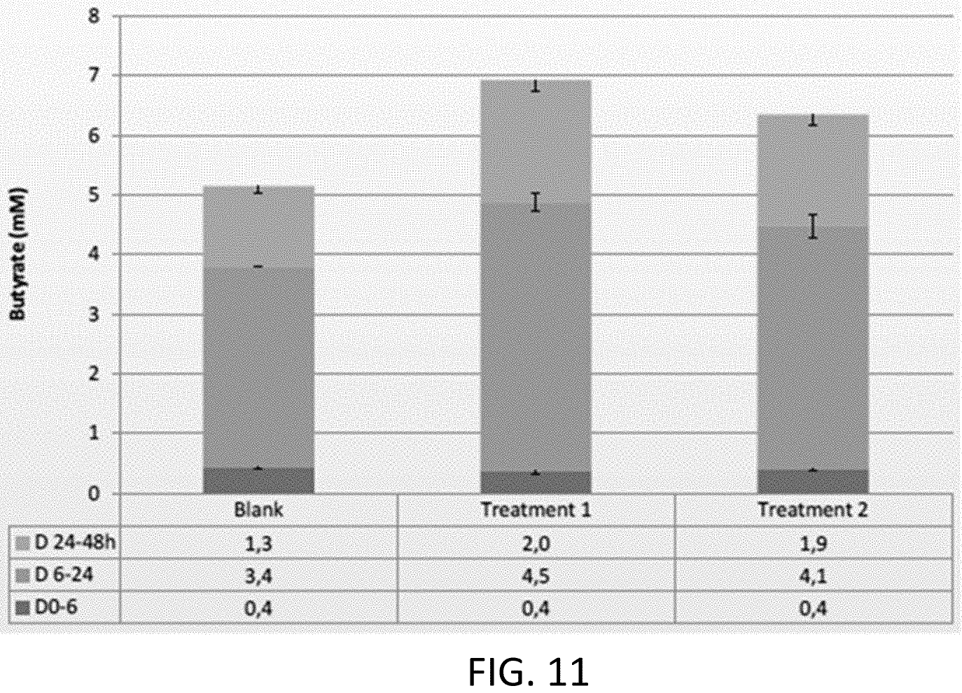

FIG. 11 illustrates butyrate production in the colon of the animal as compared to the control group in the cellulose tablet-gut-microbiome-experiment. This data shows that cellulose tablets (especially tablet 1) strongly increased butyrate production, mostly in the 6-48-hour time interval, potentially due to starch fermentation. A remarkably high production was still observed between 24-48 h (as shown in FIG. 11). Since butyrate has anti-inflammatory effects, the data indicates that consumption of cellulose tablets can help reduce inflammation in the body of the person.

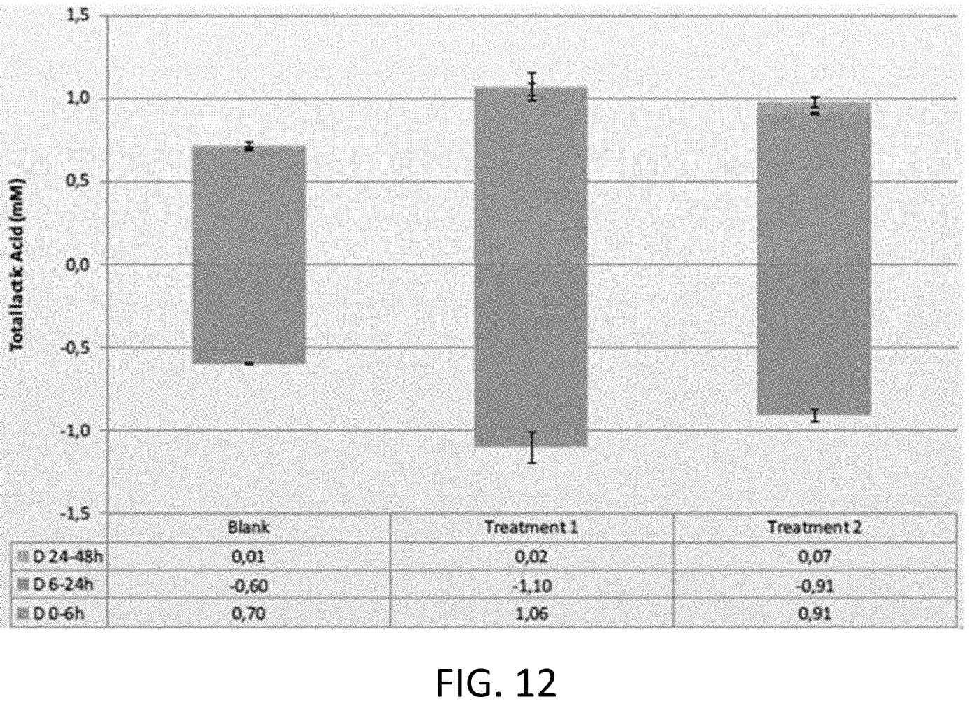

FIG. 12 illustrates lactate production and consumption in the colon of the animal as compared to the control group in the cellulose tablet-gut-microbiome-experiment. The data of FIG. 12 illustrates that cellulose tablets (especially tablet 1) increased lactate production between 0-6-hours (correlating with the stronger initial pH decrease), which was subsequently consumed in the 6-24-hour interval. This in one likely explanation for the production of propionate and butyrate (as shown in FIG. 12).

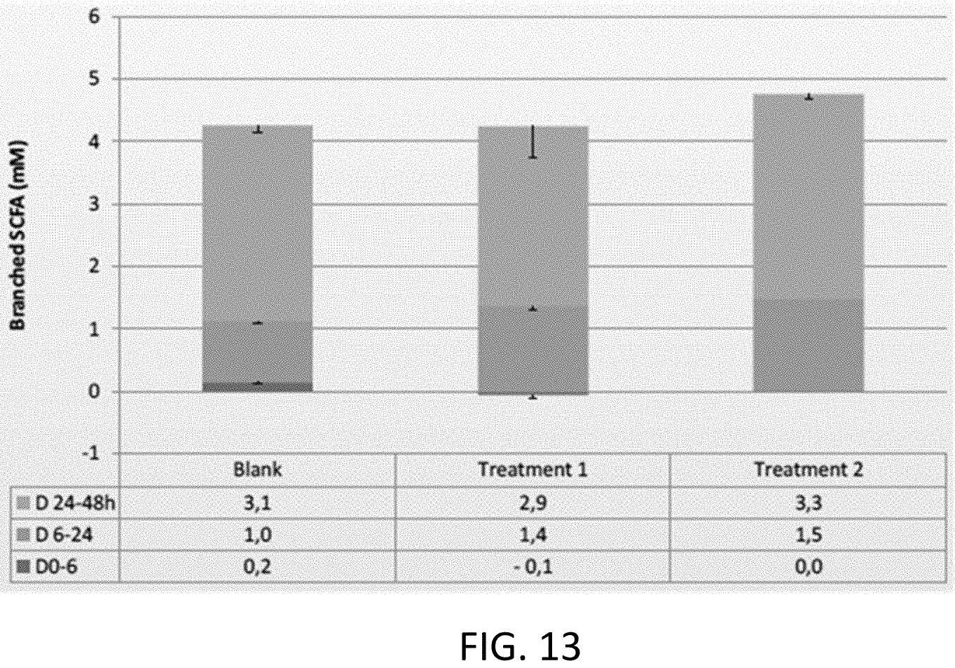

FIG. 13 illustrates branched short chain fatty acids production in the colon of the animal as compared to the control group in the cellulose tablet-gut-microbiome-experiment. The cellulose tablets 1 and 2 did not affect branched short chain fatty acids (SCFA) levels (as shown in FIG. 13). Since higher branched SCFA levels are associated with certain adverse health effects, the consumption of cellulose tablets should not increase effects associated with increased levels of branched SCFA levels in the body.

The cellulose tablet-gut-microbiome-experiment indicates that cellulose tablets 1 and 2 (with strongest effect for tablet 1) stimulated overall microbial activity, where tablets 1 had the strongest effect. Each of these tablets resulted in a stronger initial pH decrease and in a non-significant increase in gas production between 6-24-hours. The stimulated microbial activity was likely due to the delivery of starch to the colon that was converted to health-related metabolites (again with strongest effect begin noted for tablet 1). As reviewed above cellulose tablets consistent with the present disclosure may increase the production of or help provide elements to the body of an animal that are associated with positive health benefits: acetate production, probiotic administration, and acetate supplementation to among other things prevent hypertension in a model of obstructive sleep apnea (https://www.ncbi.nlm.nih.gov/pubmed/30354816).

Additionally, cellulose tablets may increase availability of or supplement elements such as propionate, butyrate, and lactate to the body to provide benefits to an animal: Propionate: The effects of dietary supplementation with inulin and inulin-propionate ester on hepatic steatosis in adults with non-alcoholic fatty liver disease (https://www.ncbi.nlm.nih.gov/pubmed/30098126); Gut-Liver axis propionate (https://www.ncbi.nlm.nih.gov/pubmed/31935110). Finally, cellulose tablets 1 and 2 did not affect the levels of metabolites that are considered as markers for protein fermentation and that are related to adverse effects on a human host.

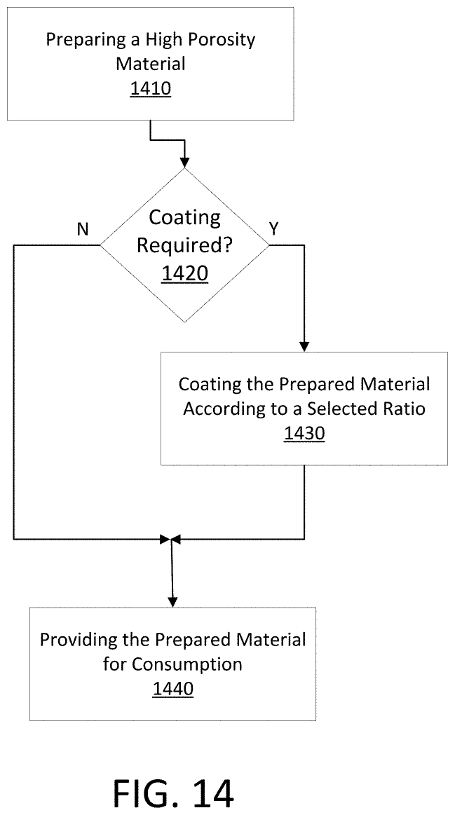

FIG. 14 illustrates steps that may be performed when a high porosity cellulose material is prepared for consumption by an animal. FIG. 14 begins with step 1410 where a high porosity cellulose material is prepared or processed into a form that may be easily consumed by animal. Step 1410 may include selecting a specific cellulose material that has pores of a size that are smaller than a size of a tablet formed from the cellulose material. Step 1410 may include chemical processing steps or mechanical steps. The cellulose material may be reduced into sizes that may easily be swallowed by an animal, for example the cellulose may be cut, chopped, or ground into small pieces of a size from the nanometer range to approximately 1 millimeter in size when forming high porosity cellulosic structures. Step 1410 may also include combining additional components selected with the group of carrageenan, glucomannan, carboxymethylcellulose, persimmon tannins, pectin, gellan gum, beta-glucan soluble fiber, psyllium husk, guar gum, locust bean gum, hydroxypropylmethylcellulose, mixed plant cell wall fibers, arabinoxylan, alginate, inulin and inulin-type fructans, high amylose RS2, galactooligosaccharide, polydextrose, resistant maltodextrin/dextrin, cross-inked phosphorylated RS4, or combinations thereof with the cellulose structure.

After the cellulose material is prepared in step 1410 process flow may move to one of at least two different steps depending on whether the prepared cellulose material will be coated as illustrated by determination step 1420 of FIG. 14. When the prepared cellulose material is coated, process flow may move to step 1430. Step 1430 may include selecting a type of coating and coating ratio (as discussed above in respect to glucomannanan or carrangeenan ratios to cellulose ratio). The cellulose material is coated, for example with one or more of carrageenan and glucomannan compounds or may be combined with a hydrogel in step 1430 of FIG. 14. After step 1430 or when the prepared cellulose material does not require a coating, program flow may move to step 1440 where the prepared material (e.g. tablet) is provided for consumption by an animal.

What is claimed is:

1. A method for treating an animal for a gut disease or condition the, method comprising providing a consumable high porosity cellulosic structure, wherein the high porosity cellulosic structure stimulates the growth of one or more microorganisms in a gut of an animal after the high porosity cellulosic structure is consumed by the animal.

2. The method of claim 1, further comprising preparing the high porosity cellulose structure from a cellulose material.

3. The method of claim 1, further comprising coating the high porosity cellulosic structure with a coating that dissolves in the animal gut based on the ph of the animal gut being greater than the ph of the mouth of the animal.

4. The method of claim 1, further comprising coating the high porosity cellulosic structure with a coating.

5. The method of claim 1 or claim 2, wherein the high porosity cellulosic structure permits diffusion of small sugars out of the cellulosic structure.

6. The method of claim 1, wherein the high porosity cellulosic structure comprises cellulose and one or more complex carbohydrate compounds selected from the group consisting of carrageenan, glucomannan, carboxymethylcellulose, persimmon tannins, pectin, gellan gum, beta-glucan soluble fiber, psyllium husk, guar gum, locust bean gum, hydroxypropylmethylcellulose, mixed plant cell wall fibers, arabinoxylan, alginate, inulin and inulin-type fructans, high amylose RS2, galactooligosaccharide, polydextrose, resistant maltodextrin/dextrin, and cross-inked phosphorylated RS4.

7. The method of claim 2, wherein the starches trapped within the high porosity cellulosic structure or the high porosity cellulosic structure itself or a portion thereof is converted to short chain fatty acids in the animal gut by the microorganisms.

8. The method of claim 1, wherein the high porosity cellulosic structure is administered prior to, subsequent to or co-administered with a probiotic.

9. A method of treating a disease or condition, the method comprising providing a consumable high porosity cellulosic structure an animal, wherein the high porosity cellulosic structure is capable of sequestering a sugar present in a gut of an animal and wherein the sequestering is thereby capable of lowering absorption of the sugar in the bloodstream of the animal.

10. The method of claim 9, wherein the high porosity cellulosic structure comprises cellulose and one or more complex carbohydrate compounds selected from the group consisting of carrageenan, glucomannan, carboxymethylcellulose, pectin, gellan gum, beta-glucan soluble fiber, psyllium husk, guar gum, locust bean gum, hydroxypropylmethylcellulose, mixed plant cell wall fibers, arabinoxylan, alginate, inulin and inulin-type fructans, high amylose RS2, galactooligosaccharide, polydextrose, resistant maltodextrin/dextrin, and cross-inked phosphorylated RS4

11. The method of claim 9, wherein the sugar comprises sucrose, fructose, glucose or any combination thereof.

12. The method according to any of claim 9, wherein an amount of sugar absorbed by the high porosity cellulosic structure in the stomach is between about 20% and 50% of the sugar present in the stomach of the animal.

13. The method of claim 9, wherein an amount of sugar absorbed by the high porosity cellulosic structure in the small intestine is between about 7% and 30% of the sugar present in the small intestine.

14. The method of claim 9, wherein an amount of sugar absorbed by the small intestine is reduced between about 3% and 10%.

15. The method according to any of claim 9, wherein the disease or condition is diabetes.

16. The method according to any of claim 9, wherein the condition is the management of glucose homeostasis.

17. A method of treating a disease or condition comprising providing a consumable high porosity cellulosic structure to an animal, wherein the high porosity cellulosic structure reduces the amount of an available component in the gut of the animal, the available component including at least one of a fat, a sugar, salt, or a high caloric food substance.

18. The method of claim 17, wherein the high porosity cellulosic structure is capable of absorbing the available component from the gut of an animal.

19. The method of claim 17, wherein the disease or condition is selected from metabolic syndrome, type 2 diabetes, non-alcoholic fatty liver disease (NAFLD), chronic inflammation, and a neuro-degenerative condition related to neuro-inflammation.

20. The method of claim 17, wherein the high porosity cellulosic structure comprises cellulose and one or more compounds selected from the group consisting of carrageenan, glucomannan and carboxymethylcellulose.

21. A composition of matter comprising a high porosity cellulosic structure prepared to a size capable of being capable of being directly swallowed by an animal, wherein the high porosity cellulosic structure includes pores that receive and entrap one or more compounds in a gut of the animal.

22. The composition of claim 23, further comprising one or more of carrageenan, glucomannan, carboxymethylcellulose, persimmon tannins, pectin, gellan gum, beta-glucan soluble fiber, psyllium husk, guar gum, locust bean gum, hydroxypropylmethylcellulose, mixed plant cell wall fibers, arabinoxylan, alginate, inulin and inulin-type fructans, high amylose RS2, galactooligosaccharide, polydextrose, resistant maltodextrin/dextrin, and cross-inked phosphorylated RS4.
